(19) United States
(12) Patent Application Publication
(10) Pub. No.: US 2013/0193280 A1
(43) Pub. Date: Aug. 1, 2013

(54) Medical Arm Support

(71) Applicant: Rodello A. Diamante, New York, NY (US)

(72) Inventor: Rodello A. Diamante, New York, NY (US)

(21) Appl. No.: 13/363,683

(22) Filed: Feb. 1, 2012

(51) Int. Cl. A61B 19/00

(52) U.S. Cl. 248/118

(57) ABSTRACT

An arm support board for supporting the arm and wrist of a medical patient with the wrist advantageously exposed for an invasive medical procedure. The arm support board may comprise a base which is generally triangular in side view, a discardable liner which mounts to the upper surface of the base to engage the arm of the patient, and discardable straps for affixing the arm of the patient to the arm support board. The straps may be passed through slots which pass through the liner. The arm support board may comprise a supplemental two part articulated support board which follows the configuration of the arm of the patient. The base may incorporate a track for slidably receiving the supplemental support board.

### FIELD OF THE INVENTION

The present invention relates to supports for medical procedures, and more particularly to a support for the wrist and arm of a patient who is to undergo an invasive cardiovascular procedure such as penetration of arm blood vessels.

### BACKGROUND OF THE INVENTION

Medical patients must occasionally have their arms and wrists supported for medical procedures such as intravenous administration of fluids, attention to injured limbs, and the like. These procedures may be somewhat prolonged, so that it becomes desirable to have a device to support the arm in a suitable position rather than have the patient consciously exert effort to maintain the desired position. Arm support boards have been proposed for accomplishing this, but have not proved entirely satisfactory as no single such product has come into widespread use in the medical field.

One of the issues that confront arm support boards is compatibility with individual physiology. An arm support board should be reasonably adjustable to the individual so that many different sizes need not be maintained in inventory of medical facilities.

Also, issues such as the ability to discard medical devices which have been exposed to bodily fluids for example impose new demands on devices such as arm support boards. The issue of prevention of cross contamination must be addressed. Also, it is desirable to have the medical field engage in environmentally friendly practices, such as fabricating discardable items from readily biodegradable materials.

### SUMMARY OF THE INVENTION

The present invention sets forth an arm support board which addresses the issues raised above. The novel arm support board comprises a base configured to establish a suitably configured support platform for holding the forearm and wrist in a suitable position, a liner which may rest on and cover the base and receive the arm of the patient, and straps adapted to suitably secure the arm to the arm support board. The arm support board may comprise a slidably received articulated arm engaging panel which is adjustable to the upper arm and forearm of the patient.

The base may be formed from a durable material to enable repeated uses. The liner may be discardable, being formed from cardboard for example.

It is an object of the invention to provide a practical arm support board which is adaptable to different patients, incorporates inexpensive, discardable components, and is easy to prepare for medical procedures.

It is an object of the invention to provide improved elements and arrangements thereof by apparatus for the purposes described which is inexpensive, dependable, and fully effective in accomplishing its intended purposes.

These and other objects of the present invention will become readily apparent upon further review of the following specification and drawings.

### BRIEF DESCRIPTION OF THE DRAWINGS

Various objects, features, and attendant advantages of the present invention will become more fully appreciated as the same becomes better understood when considered in conjunction with the accompanying drawings, in which like reference characters designate the same or similar parts throughout the several views, and wherein:

FIG. 1 is a perspective view of an arm support board according to at least one aspect of the invention, shown fully assembled and ready to receive the arm of a patient.

FIG. 2 is a partially exploded view of FIG. 1.

FIG. 3 is an exploded side view of two of the components of FIG. 1.

FIG. 4 is an exploded end view of FIG. 3.

FIG. 5 is a perspective view of a strap seen near the center of FIG. 1.

### DETAILED DESCRIPTION

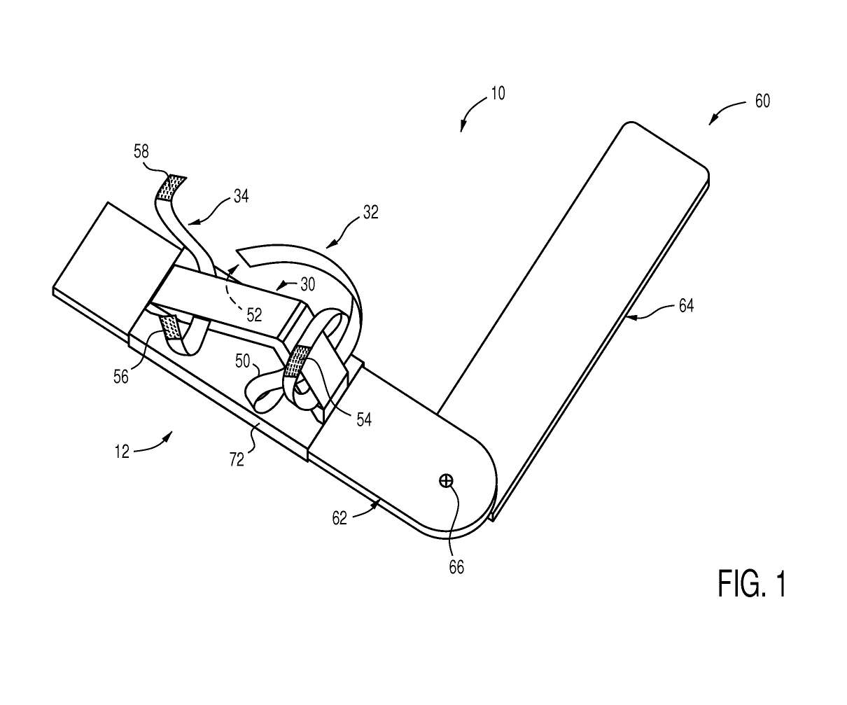

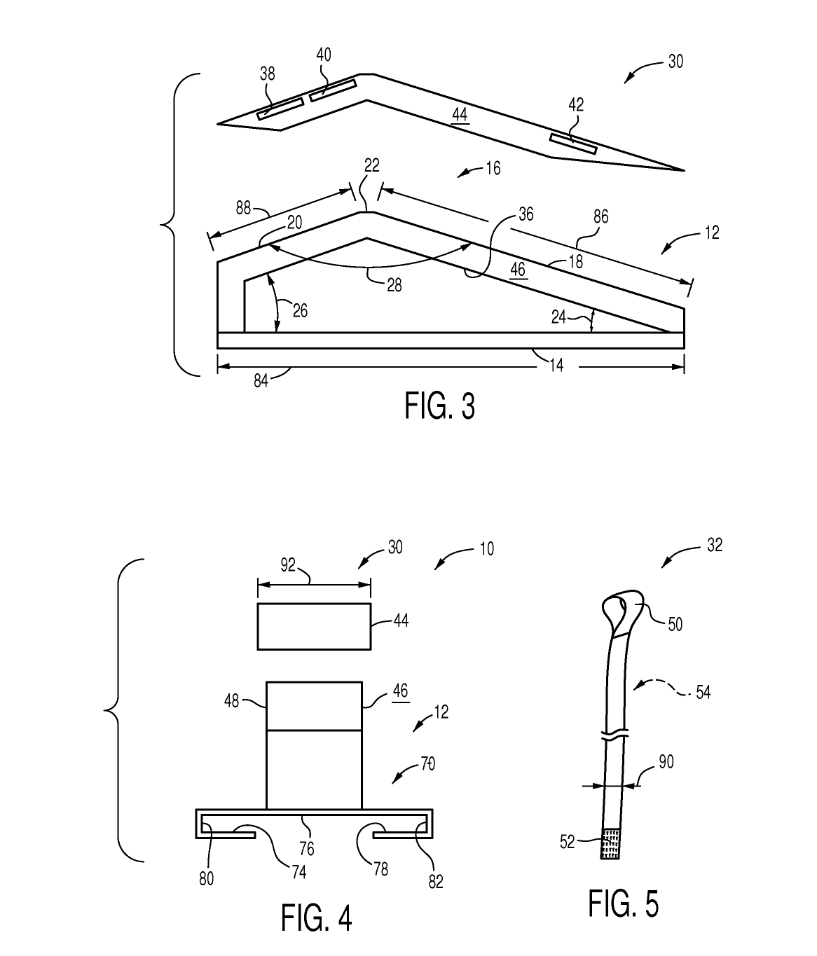

Referring first to FIGS. 1 and 3, according to at least one aspect of the invention, shows an arm support board 10 for supporting the arm and wrist of a medical patient (not shown) on a generally horizontal environmental surface (not shown) such as a bench or tabletop. In some instances, depending upon the nature of the medical procedure being undertaken, it would be possible to support the arm on a surface which is not strictly speaking horizontal, and which may not be fixed in spatial orientation, as a bench or tabletop may be presumed to be. For example, the patient's arm could be supported on his or her lap, or be held by another person. Therefore, while ordinarily contemplated practice is to utilize a horizontal environmental surface, the term “horizontal” will be understood to be a semantic convenience for purposes of description of the invention.

It should be noted at this point that orientational terms such as upward and downward refer to the subject drawing as viewed by an observer. The drawing figures depict their subject matter in orientations of normal use, which could obviously change with changes in body posture and position of the patient. Therefore, orientational terms must be understood to provide semantic basis for purposes of description, and do not limit the invention or its component parts in any particular way.

The arm support board 10 supports the arm and wrist such that the arm is inclined from the elbow to the wrist, with the wrist advantageously exposed for an invasive medical procedure such as intravenous administration of fluids, among others requiring that the wrist be suitably exposed and stabilized. The arm support board 10 comprises a base 12 serving as a structural member which bears the weight of the arm of the patient and also determines orientation of the arm and wrist relative to the horizontal surface. The base 12 comprises a bottom surface 14 for resting on or engaging the horizontal environmental surface and an upper support surface 16 for receiving the forearm, wrist, and hand of the patient. The upper support surface 16 is disposed to define a forearm ramp 18, a wrist ramp 20, and optionally, a transition zone 22 adapted to make transition from the forearm ramp 18 and the wrist ramp 20. The transition may assure that the upper support surface 16 remain continuous from the forearm ramp 18 to the wrist ramp 20, and may be configured to eliminate sharp corners, edges, and the like which could result in discomfort to the patient. The important aspect of the forearm ramp 18 and of the wrist ramp 20 is the theoretical support platform or plane established by their respective upper surfaces, which respective upper surfaces comprise the substantial and significant portion of the upper support surface 16. Therefore, the terms “forearm ramp” and “wrist ramp” will be understood to refer either to the complete structural members formed by each, or merely to their respective upper surfaces, depending upon context. It should be noted that although described in terms of planes, the upper support surface 16 may not literally form planes. Also, the support surface 16 need not be continuous along its extent. For example, the upper support surface 16 could have openings formed therein, provided that sufficient surface is present to stably support the arm of the patient.

The upper surface of the forearm ramp 18 is disposed at an acute angle 24 to a horizontal direction when the bottom surface 14 of the base 12 engages the horizontal environmental surface. The upper surface of the wrist ramp 20 is disposed at a second acute angle 26 to the horizontal direction when the bottom surface 14 of the base 12 engages the horizontal environmental surface. Deviation of the plane generally provided by the upper surface of the forearm ramp 18 from the horizontal direction is less than corresponding deviation of the wrist ramp 20 from the horizontal direction. The forearm ramp 18 and the wrist ramp 20 are inclined in opposed directions such that the upper support surface 16 when considered in combination with the bottom surface 14 generally creates a triangular visual impression when viewed from the side as in FIG. 3.

The upper surfaces of the forearm ramp 18 and the wrist ramp 20 collectively define a downwardly facing included angle 28 therebetween when the bottom surface 14 of the base 12 engages the horizontal surface, as it would in ordinary use. Magnitude of this downwardly facing included angle 28 is between one hundred forty and one hundred fifty degrees, more preferably between one hundred forty-four and one hundred forty-eight degrees, and most preferably about one hundred forty-six degrees.

The base 12 is preferably fabricated from a durable constituent material, such as a sturdy polymer or from metallic aluminum or an alloy of aluminum.

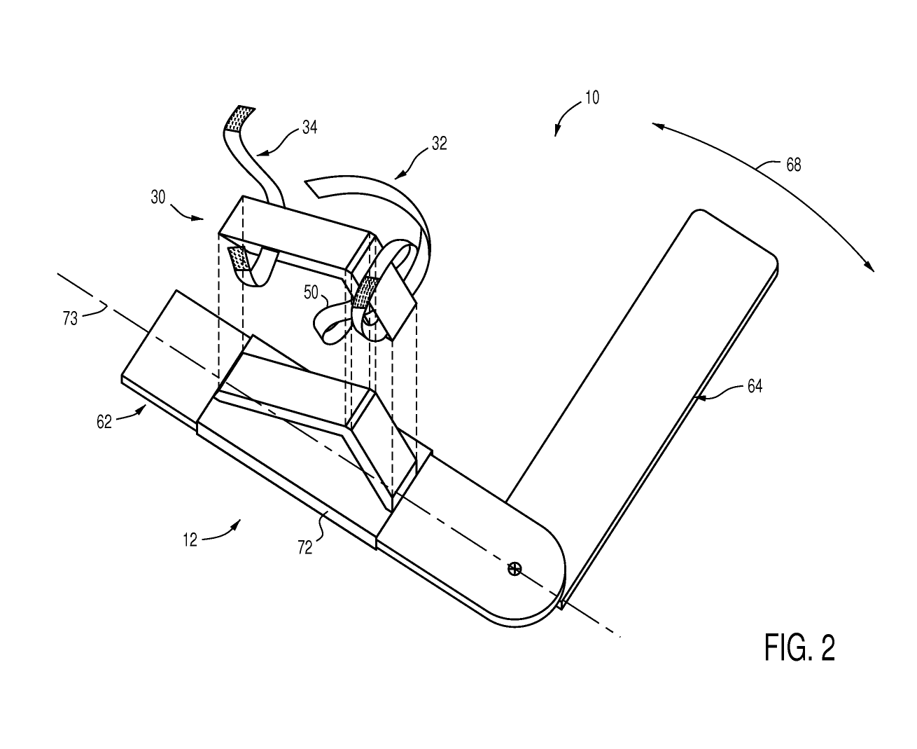

The arm support board 10 also comprises a liner 30 for covering the base 12, wherein the liner 30 is configured to cooperate with, cover, and slightly overlap the base 12 when the liner 30 is placed onto the base 12 from above as seen in FIG. 2, FIG. 1 showing the liner 30 installed on the base 12. The liner 30 serves as a barrier between the base 12 and the arm of the patient, for preventing contact and transfer of contamination between the arm and the base 12. The liner 30 is substantially fabricated from an inexpensive constituent material such as a natural cellulosic paper for example, and therefore is discardable. As employed herein, the term “paper” will be understood to encompass both thick and thin paper-like materials, and may include cardboard, both solid and corrugated, paperboard, and similar materials of limited flaccidity. Constituency of the liner 30 is not necessarily tied to natural cellulosic materials, that being merely one of the best known of many possible materials. Other materials including blends or combinations of materials may be utilized in place of pure natural cellulosic materials. If desired, the liner 30 may be flaccid, such as being fabricated from a woven or non-woven fabric or from a suitable polymer (these embodiments are not shown).

The arm of the patient must be secured to a degree to the base 12 and the liner 30 to achieve necessary stability to enable the medical procedure to be satisfactory. Straps 32, 34 may be provided for temporarily retaining the arm of a patient on the arm support platform throughout the course of the medical procedure. The straps 32, 34 may be removably attachable to the base 12. Although this may be accomplished in a number of ways, one way is to pass each strap 32 or 34 through an open interior 36 located below the upper surface 16 and above the bottom surface 14 of the base 12, as best seen in FIG. 3.

Also clearly visible in FIG. 3 are slots 38, 40, and 42 formed in the liner 30, which slots 38, 40, 42 are each dimensioned and configured to receive and retain one of the straps 32 or 34. These slots 38, 40, 42 are intended to extend entirely through the liner 30 so that the straps 32, 34 may be passed through a slot 38, 40, or 42, through the open interior 36 of the base 12, and if necessary through additional slots (not shown) so that the straps 32, 34 may be in a position to be fastened over the arm of the patient, as seen in FIG. 1. Although it would be possible to form the liner 30 as a solid member wherein each slot 38, 40, or 42 passes entirely through the liner 30, the same result may be achieved when the liner 30 is configured to include depending side walls such as the side wall 44. The latter construction is preferred, so that when installed as shown in FIG. 1, the liner 30 contacts the base 12 along the upper support surface 16 and also along lateral walls 46 and 48 (see FIG. 4) of the base 12. The same result, that of providing a path for insertion of the straps 32, 34 entirely through the liner 30, is achieved by providing corresponding slots (not visible in the side view of FIG. 3) on the lateral wall 48 at the opposite side of the lateral wall 46.

The slots 38, 40 are formed in close proximity to one another at that portion of the liner 30 which after assembly as seen in FIG. 1 will result in the slots 38, 40 being at the wrist ramp 20. The slot 42 is formed at that portion of the liner 30 such that after assembly as seen in FIG. 1 the slot 42 is at the forearm ramp 18.

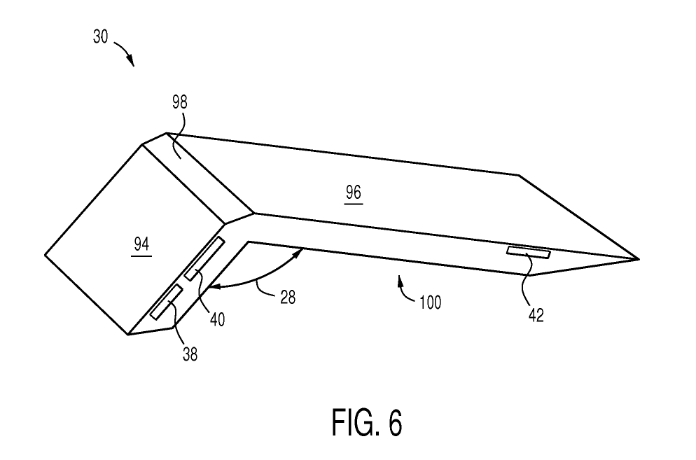

Although it would be possible to provide only one slot, such as the slots 40 and 42, for the two straps 32, 34, it is preferred to provide the two slots 38, 40 for the strap 32 so that the strap 32 may be passed twice through the base 12 and the liner 30, as seen in FIG. 2. The two slots 38, 40 enable the strap 32 to be passed twice through the liner 30 at the wrist ramp 20 and to be retained proximate the wrist ramp 20. The reason for passing the strap 32 twice through the base 12 or liner 30 is to stably enable the strap 32 to engage the thumb of the patient by a closed loop 50 at which the strap 32 terminates, and which is dimensioned and configured to receive and retain the thumb of the patient when the arm of the patient is strapped to the arm support board 10. The strap 32 is illustrated as it would be if drawn taut in FIG. 5.

Each one of the straps 32, 34 may have a suitable fastener such as complementary patches 52, 54 and 56, 58 of hook and loop fastening material. Complementary patches are those including two different polarities of fastening material (i.e., one patch of barbs or hooks, and the other patch of loops or pile). The patches 52, 54 and 56, 58 may be located on their respective straps 32, 34 such that the straps 32, 34 may each be fastened to themselves upon overlapping themselves in known fashion.

Referring again to FIG. 1, the arm support board 10 may further comprise a supplementary board 60 which may be utilized to further stabilize the arm of the patient. The supplementary board 60 may be formed to include a first section 62 and a second section 64 pivotally coupled to one another at a swivel joint 66 to enable the second section to pivot about the swivel joint 66 as indicated by an arcuate arrow 68. The supplementary board 60 may be interfittably compatible with a track 72 formed in the base 12 in the following way.

Both the first section 62 (as shown) and the second section 64 of the supplementary board 60 may be slidably coupled to the base 12 by a connector 70 formed as part of the base 12. The first section 62 may be inserted into the connector 70 and slidably adjusted therealong in a direction coinciding with the longitudinal axis 73 of the base 12 (called out in FIG. 2) to enable adjustment of position of the supplementary board 60 in the direction of the length of the forearm of the patient when the forearm of the patient is secured to the arm support board 10 in the operable position.

The connector 70, which may be disposed at the bottom surface 14 of the base 12, may comprise a track which partially surrounds the supplementary support board 60, as seen in FIG. 1. This will also be apparent in FIG. 4, where upper and lower sliding contact surfaces 74, 76, 78 and lateral surfaces 80, 82 constrain the supplementary board 60 to move only parallel to the longitudinal axis 73.

Turning now to FIG. 3, the overall length (indicated by an arrow 84) of the base may be between ten and fifteen inches, and more preferably about twelve inches. The effective length (indicated by an arrow 86) of the forearm ramp 18 may be between seven and ten inches, and more preferably about eight and one quarter to eight and one half inches. The wrist ramp 20 may have a length (indicated by an arrow 88) of between three and five inches, and more preferably about three and three quarter inches to four inches. The slots 38, 40, and 42 may be just large enough to pass the straps 32, 34. An exemplary width (indicated by an arrow 90 in FIG. 5) of the straps 32, 34 is just under one inch.

Other dimensions of the arm support board 10 may be extrapolated from the drawings, which are generally drawn to scale apart from the right-to-left width (indicated by an arrow 92) of the liner 30, as shown in FIG. 4. This width is potentially exaggerated in FIG. 4 to emphasize the overlapping dimensions of the liner 30 relative to the base 12. Of course, the arm support board 10 is only representative of many different embodiments which may be realized. Hence, dimensions determined by extrapolating may possibly apply only to the embodiment of the invention represented by the arm support board 10.

While the present invention has been described in connection with what is considered the most practical and preferred embodiment, it is to be understood that the present invention is not to be limited to the disclosed arrangements, but is intended to cover various arrangements which are included within the spirit and scope of the broadest possible interpretation of the appended claims so as to encompass all modifications and equivalent arrangements which are possible.

I claim:

1. An arm support board for supporting the arm and wrist of a medical patient on a horizontal environmental surface such that the arm is inclined from the elbow to the wrist, with the wrist advantageously exposed for an invasive medical procedure, comprising:
a base comprising a bottom surface for engaging the horizontal environmental surface, an upper support surface disposed to define a forearm ramp disposed at a first acute angle to a horizontal direction when the bottom surface of the base engages the horizontal environmental surface and a wrist ramp disposed at a second acute angle to the horizontal direction when the bottom surface of the base engages the horizontal environmental surface, wherein the forearm ramp and the wrist ramp are inclined in opposed directions and wherein deviation of the forearm ramp from the horizontal direction is less than deviation of the wrist ramp from the horizontal direction;
a liner for covering the base, wherein the liner is configured to cooperate with, cover, and slightly overlap the base when the liner is placed onto the base from above; and
at least one strap for temporarily retaining the arm of a patient on the arm support platform, which said strap is removably attachable to the base.

2. The arm support board of claim 1, wherein the liner is discardable.

3. The arm support board of claim 1, wherein the liner is substantially fabricated from a natural cellulosic paper constituent material.

4. The arm support board of claim 1, wherein the liner comprises at least one slot extending entirely through the liner, wherein the slot is dimensioned and configured to receive and retain one of the straps.

5. The arm support board of claim 1, wherein the arm support board comprises at least two straps and the liner comprises at least two slots each dimensioned and configured to receive and retain one of the straps.

6. The arm support board of claim 5, wherein one of the slots is formed at the forearm ramp and another one of the slots is formed at the wrist ramp.

7. The arm support board of claim 5, wherein one of the slots is formed in the liner at the forearm ramp and two other slots are formed in the liner in close proximity to one another at the wrist ramp in a manner enabling one of the straps to be passed twice through the wrist ramp and to be retained proximate the wrist ramp.

8. The arm support board of claim 5, wherein one of the straps terminates at a closed loop which is dimensioned and configured to receive and retain the thumb of a patient when the arm of the patient is strapped to the arm support board.

9. The arm support board of claim 1, wherein the base is fabricated from a durable constituent material.

10. The arm support board of claim 9, wherein the durable constituent material comprises a polymer.

11. The arm support board of claim 9, wherein the durable constituent material comprises metallic aluminum.

12. The arm support board of claim 1, wherein the strap has at least two patches of hook and loop fastening material of opposed polarity arranged such that the strap can be fastened to itself.

13. The arm support board of claim 1, wherein the base comprises an open interior located below the upper surface and above the bottom surface of the base.

14. The arm support board of claim 1, wherein the base has a length between ten and fifteen inches, the forearm ramp has a length between seven and ten inches, and the wrist ramp has a length between three and five inches.

15. The arm support board of claim 1, wherein the forearm ramp and the wrist ramp have a downwardly facing included angle therebetween when the bottom surface of the base engages the horizontal surface, and the downwardly facing included angle is between one hundred forty-four and one hundred forty-eight degrees.

16. The arm support board of claim 1, wherein the forearm ramp and the wrist ramp have a downwardly facing included angle therebetween when the bottom surface of the base engages the horizontal surface, and the downwardly facing included angle is about one hundred forty-six degrees.

17. The arm support board of claim 1, further comprising a connector disposed at the bottom surface of the base, wherein the connector is disposed to couple to a supplementary support board and to enable adjustment of position of the supplementary support board in the direction of the length of the forearm of a patient when the forearm of the patient is secured to the arm support board in the operable position.

18. The arm support board of claim 17, wherein the connector comprises a track which partially surrounds the supplementary support board and is disposed to enable slidable adjustment of the supplementary support board along the base.

19. The arm support board of claim 18, further comprising a supplementary support board which is interfittingly compatible with the track.

20. The arm support board of claim 19, wherein the supplementary support board comprises a first section and a second section which is pivotally coupled to the first section.
